(19) United States
(12) Patent Application Publication
(10) Pub. No.: US 2005/0244973 A1
(43) Pub. Date: Nov. 3, 2005

(54) Biological patterns for diagnosis and treatment of cancer

(75) Inventors: Frank Andel III, Berkeley, CA (US); Hans Bitter, San Francisco, CA (US); Mikhail Belov, Burlingame, CA (US); Michael Brown, San Francisco, CA (US); Alfred C. Greenquist, San Jose, CA (US); Jonathan C. Heller, San Francisco, CA (US); Alexander Sassi, Berkeley, CA (US); John T. Stults, Redwood City, CA (US); Kathy Stults, Redwood City, CA (US)

Correspondence Address: WILSON SONSINI GOODRICH & ROSATI, 650 PAGE MILL ROAD, PALO ALTO, CA, 94304-1050, US

(73) Assignee: Predicant Biosciences, Inc., South San Francisco, CA (US)

(21) Appl. No.: 10/845,773

(22) Filed: May 14, 2004

Related U.S. Application Data
(60) Provisional application No. 60/566,956, filed on Apr. 29, 2004.

(51) Int. Cl. G01N 33/48

(52) U.S. Cl. 436/64; 436/173

(57) ABSTRACT

The present invention provides methods for diagnosing cancers, such as prostate cancer. Also, methods for evaluating the prostate cancer state of a patient are described herein. These methods involve the detection, analysis, and classification of biological patterns in biological samples. The biological patterns are obtained using, for example, mass spectrometry systems, antibody based techniques, or nucleic acid based techniques. The present invention also includes therapeutic and prophylactic agents that target the biomarkers described herein. Also, the present invention provides methods for the treatment of prostate cancer using the markers described herein or agents that mimic the properties of these markers.

### CROSS-REFERENCE TO RELATED APPLICATIONS

This application claims the benefit of U.S. Provisional Application No. ______ filed Apr. 29, 2004, WSGR Docket No. 29191-719.101, which is incorporated herein by reference.

### BACKGROUND OF THE INVENTION

Cancers are a complex set of diseases that result from genetic alterations both inherited and accrued over the lifetime of the individual. These genetic changes give rise to molecular alterations that distinguish cancer cells from normal cells. The number and type of alterations underlying cancers vary not only between cancers but also over the progression of the cancer and even within individual cancers. This results in an enormous diversity of phenotypes, especially at the molecular level, and corresponds with the observed diversity in path of progression, outcome, and response to therapy of various cancers, even when they have common presentation.

The current inability to distinguish between cancers, or to predict their prognosis and likely response to treatment, is a result of the inability to adequately identify and assess the biological state of an individual. This is reflected in the limited ability to detect the earliest stages of disease (e.g. stage I cancer detection), anticipate the path any apparent disease will take in one patient versus another (e.g. metastasis or remission prediction), predict the likelihood of response for any individual to a particular treatment (e.g. adjuvant and neo-adjuvant chemotherapeutic responses), and preempt the possible adverse effects of treatments on a particular individual (e.g. monitoring toxicology due to chemotherapy). New technologies and strategies are needed to define biological states related to cancer and thereby inform medical care and improve the repertoire of medical tools to treat cancer patients.

### BRIEF SUMMARY OF THE INVENTION

One aspect of the present invention is methods for the diagnosis of cancer, such as prostate cancer. In one embodiment, prostate cancer states are analyzed using the prostate cancer markers described herein. These markers can be detected using mass spectrometry, antibody based techniques, nucleic acid based techniques, or any other suitable technique known in the art.

Another aspect of the invention includes prostate cancer therapeutic agents that modulate the markers described herein. In one embodiment, the markers themselves or agents that mimic their properties are used in the treatment of prostate cancer.

### BRIEF DESCRIPTION OF THE FIGURES

FIG. 1 is a schematic representation of the experimental design.

FIG. 2 is a schematic representation of the cancer pooling procedure.

FIG. 3 is a flowchart illustrating an embodiment of a method of the invention.

FIG. 4 is a flowchart illustrating an embodiment of a method of the invention.

FIG. 5 depicts an apparatus suitable for use in the methods of the invention.

FIG. 6 illustrates an apparatus suitable for use in the methods of the invention.

### DETAILED DESCRIPTION OF THE INVENTION

In one aspect, the present invention provides methods for diagnosing prostate cancer. Also, methods for evaluating the prostate cancer state of a patient are described herein. These methods involve the detection, analysis, and classification of biological patterns in biological samples. Biological patterns are typically composed of signals from markers such as, but not limited to, proteins, peptides, protein fragments, small molecules, sugars, lipids, fatty acids, or any other component found in a biological sample. The term “protein” as used herein refers to an organic compound comprising two or more amino acids covalently joined by peptide bonds. Proteins include, but are not limited to, peptides, oligopeptides, glycosylated peptides, and polypeptides. The biological patterns used in the present invention are typically patterns of markers. Preferably, the markers identified and used in the present invention are prostate cancer markers. The terms “markers” and “biomarkers” are used herein interchangeably. It is preferred that the biological patterns comprise signals from one or more proteins. Preferably the number of markers in these patterns can be more than about 5, more preferably more than about 25, even more preferably more than about 50, and even more preferably more than about 100. In some embodiments, the markers being analyzed do not include glycolipids or oligosaccharides.

In preferred embodiments, the biological patterns are obtained using mass spectrometry systems. Some embodiments are mass spectrometry systems that do not involve the use of protein affinity chips, for example chips with specific or non-specific binding surfaces (e.g. hydrophobic surfaces). In some embodiments, the samples are prepared and separated with fluidic devices, preferably microfluidic devices, and delivered to the mass spectrometry system by electrospray ionization (ESI). In some embodiments, the delivery happens “on-line”, e.g. the separations device is directly interfaced to a mass spectrometer and the spectra are collected as fractions move from the column, through the ESI interface into the mass spectrometer. In other embodiments, fractions are collected from the separations device (e.g. “off-line”) and those fractions are later run using direct-infusion ESI mass spectrometery. In yet another embodiment, the samples are prepared and separated with fluidic devices, preferably microfluidic devices, and spotted on a MALDI plate for laser-desorption ionization.

The identification and analysis of cancer markers, especially prostate cancer markers, have numerous therapeutic and diagnostic purposes. Clinical applications include, for example, detection of disease; distinguishing disease states to inform prognosis, selection of therapy, and/or prediction of therapeutic response; disease staging; identification of disease processes; prediction of efficacy of therapy; monitoring of patients trajectories (e.g., prior to onset of disease); prediction of adverse response; monitoring of therapy associated efficacy and toxicity; and detection of recurrence. Also, these cancer markers can be used in assays to identify novel therapeutics. In addition, the markers can be used as targets for cancer drugs, especially prostate cancer drugs, and therapeutics, for example antibodies against the markers or fragments of the markers can be used as prostate cancer therapeutics. The present invention also includes therapeutic and prophylactic agents that target the biomarkers described herein. In addition, the markers can be used as prostate cancer drugs or therapeutics themselves.

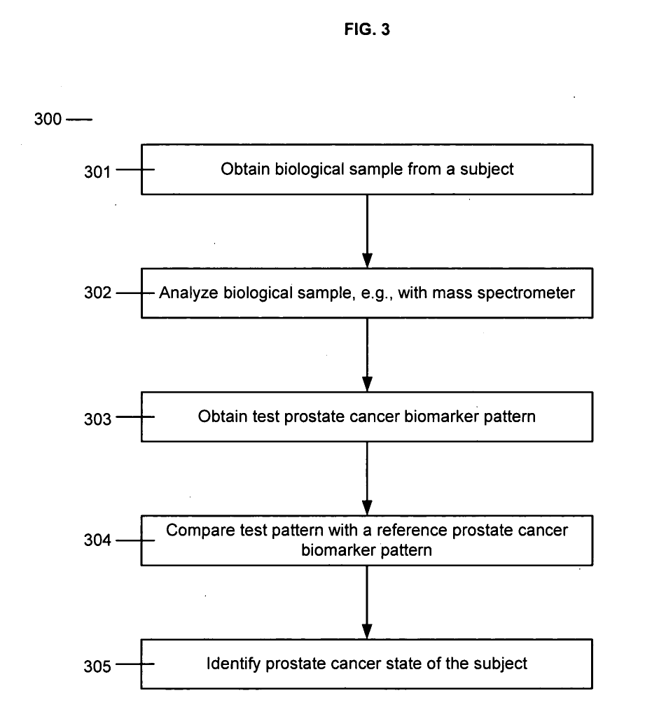

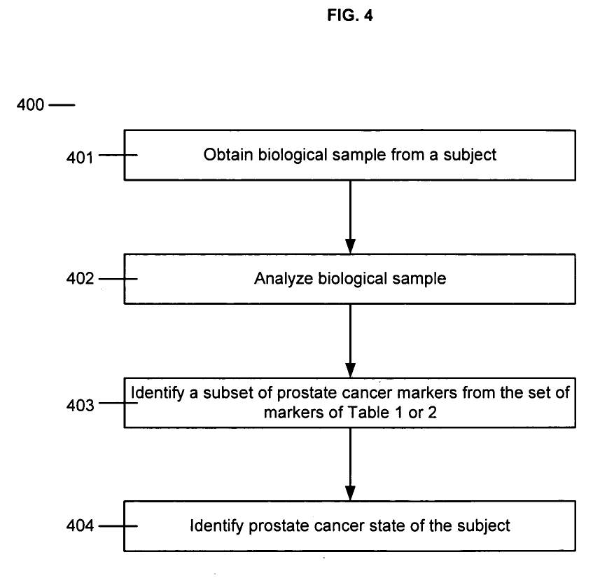

Two embodiments of the methods of the present invention are depicted in FIGS. 3 and 4. In one embodiment, a biological sample is obtained from a subject, preferably a human, at step 301. The sample is analyzed with a mass spectrometer at step 302. A test biomarker pattern is obtained for the subject at step 303) and this test pattern is compared with a reference pattern at step 304. Based on this comparison a decision is made regarding the cancer state, such as the prostate cancer state, of the subject. Preferably, the test and reference patterns are protein patterns. The reference pattern may be obtained from the same subject or from a different subject who is either not affected with the disease or is a prostate cancer patient. The reference pattern could be obtained from one subject or multiple subjects. In another embodiment, a biological sample is obtained from a subject at step 401. The biological sample is analyzed at step 402 and the analysis is conducted using a technique suitable for identifying one or more cancer markers of Table 1 and/or Table 2. The prostate cancer markers are identified at step 403 and based on this identification a decision is made regarding the prostate cancer state of the subject at step 404.

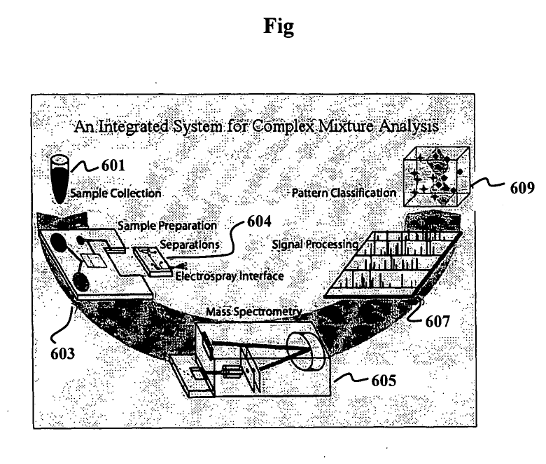

FIG. 6 illustrates an exemplary system platform suitable for use herein. Biological fluids 601 include but are not limited to serum, plasma, whole blood, nipple aspirate, pancreatic fluid, trabecular fluid, lung lavage, urine, cerebrospinal fluid, saliva, sweat, pericrevicular fluid, and tears. The system provides for the integration of fast molecular separations and electrospray ionization system 604 on a microfluidics platform 603. The system provides processed samples to a high sensitivity time of flight mass spectrometer 605. Signal processing system and pattern extraction and recognition tools 605 incorporate domain knowledge to extract information from biomarker patterns and classify the patterns to provide a classification 609. The microfluidics device(s) 603 may be formed in plastic by means of etching, machining, cutting, molding, casting or embossing. The microfluidics device(s) may be made from glass or silicon by means of etching, machining, or cutting. The device may be formed by polymerization on a form or other mold. The molecular separations unit or the integrated fast molecular separations/electrospray ionization unit may provide additional sample preparation steps, including sample loading, sample concentration, removal of salts and other compounds that may interfere with electrospray ionization, removal of highly abundant species, proteolytic or chemical cleavage of components within the biological material, and/or aliquoting in to storage containers.

Methods and Systems for Determining Patterns of Cancer Markers

Collection and Preparation of Biological Sample

Biological samples are obtained from individuals with varying phenotypic states, particularly various states of prostate cancer. Examples of phenotypic states also include phenotypes of a non-cancerous state, which is typically used for comparisons to prostate cancer states. Other examples of phenotypic states include other prostate diseases or other cancers. In a preferred embodiment, examples of various phenotypic states of prostate cancer are matched with control samples that are obtained from individuals who do not exhibit the phenotypic state of prostate cancer (e.g., an individual who is not affected by a disease).

Samples may be collected from a variety of sources in a given patient. Samples collected are preferably bodily fluids such as blood, serum, sputum, including, saliva, plasma, nipple aspirants, synovial fluids, cerebrospinal fluids, sweat, urine, fecal matter, pancreatic fluid, trabecular fluid, cerebrospinal fluid, tears, bronchial lavage, swabbings, bronchial aspirants, semen, prostatic fluid, precervicular fluid, vaginal fluids, pre-ejaculate, etc. In a preferred embodiment, a sample collected is approximately 1 to approximately 5 ml of blood. In another preferred embodiment, a sample collected is approximately 10 to approximately 15 ml of blood.

In some instances, samples may be collected from individuals repeatedly over a longitudinal period of time (e.g., once a day, once a week, once a month, biannually or annually). Obtaining numerous samples from an individual over a period of time can be used to verify results from earlier detections and/or to identify an alteration in biological pattern as a result of, for example, drug treatment, etc. Samples can be obtained from humans or non-humans. In a preferred embodiment, samples are obtained from humans.

Sample preparation and separation can involve any of the following procedures, depending on the type of sample collected and/or types of biological molecules searched: removal of high abundance polypeptides (e.g., albumin, gamma globulin, and transferring, etc.); addition of preservatives and calibrants, addition of protease inhibitors, addition of denaturants, desalting of samples; concentration of sample proteins; protein digestions; and fraction collection. The sample preparation can also isolate molecules that are bound in non-covalent complexes to other protein (e.g., carrier proteins). This process may isolate only those molecules bound to a specific carrier protein (e.g., albumin), or use a more general process, such as the release of bound molecules from all carrier proteins via protein denaturation follow by removal of the carrier proteins. Preferably, sample preparation techniques concentrate information-rich proteins (e.g., proteins that have “leaked” from diseased cells) and deplete proteins that would carry little or no information such as those that are highly abundant or native to serum

Sample preparation can take place in a multiplicity of devices including preparation and separation devices or on a combination preparation/separation device. In a preferred embodiment, such preparation/separation device is a microfluidics device. Optimally, the preparation/separation device interfaces directly or indirectly with a detection device. In another embodiment, such preparation/separation device is a fluidics device.

Approximately 100 μL of a sample is analyzed per assay in some embodiments of the invention. Removal of undesired proteins (e.g., high abundance, uninformative, or undetectable proteins) can be achieved using high affinity reagents, high molecular weight filters, ultracentrifugation and/or electrodialysis. High affinity reagents include antibodies or other reagents (e.g. aptamers) that selectively bind to high abundance proteins. Sample preparation could also include ion exchange chromatography, metal ion affinity chromatography, gel filtration, hydrophobic chromatography, chromatofocusing, adsorption chromatography, isoelectric focusing and related techniques. Molecular weight filters include membranes that separate molecules on the basis of size and molecular weight. Such filters may further employ reverse osmosis, nanofiltration, ultrafiltration and microfiltration.

Ultracentrifugation is another method for removing undesired polypeptides. Ultracentrifugation is the centrifugation of a sample at about 60,000 rpm while monitoring with an optical system the sedimentation (or lack thereof) of particles. Finally, electrodialysis is a procedure which uses an electromembrane or semipermable membrane in a process in which ions are transported through semi-permeable membranes from one solution to another under the influence of a potential gradient. Since the membranes used in electrodialysis may have the ability to selectively transport ions having positive or negative charge and reject ions of the opposite charge, or to allow species to migrate through a semipermable membrane based on size and charge, electrodialysis is useful for concentration, removal, or separation of electrolytes.

In a preferred embodiment, the manifold or microfluidics device performs electrodialysis to remove high molecular weight polypeptides or undesired polypeptides. Electrodialysis is first used to allow only molecules under approximately 30 kD (not a sharp cutoff) to pass through into a second chamber. A second membrane with a very small molecular weight cut-off (roughly 500-1000 D) will allow smaller molecules to egress the second chamber.

In a preferred embodiment, the manifold or microfluidics device performs electrodialysis to remove high molecular weight polypeptides or undesired polypeptides. Electrodialysis is first used to allow only molecules under approximately 30 kD (not a sharp cutoff) to pass through into a second chamber. A second membrane with a very small molecular weight cut-off (roughly 500 D) will allow smaller molecules to egress the second chamber.

After samples are prepared, components that may comprise a biological pattern of interest may be separated. Separation can take place in the same location as the preparation or in another location. In a preferred embodiment, separation occurs in the same microfluidics device where preparation occurs, but in a different location on the device. Samples can be removed from an initial manifold location to a microfluidics device using various means, including an electric field. In a preferred embodiment, the samples are concentrated during their migration to the microfluidics device using reverse phase beads and an organic solvent elution such as about 50% methanol. This can elute the molecules into a channel or a well on a separation device of a microfluidics device.

Separation can involve any procedure known in the art, such as capillary electrophoresis (e.g., in capillary or on-chip) or chromatography (e.g., in capillary, column or on a chip).

Electrophoresis is a method which can be used to separate ionic molecules such as polypeptides according to their mobilities under the influence of an electric field. Electrophoresis can be conducted in a gel, capillary, or in a microchannel on a chip. In a capillary or microchannel, the mobility of a species is determined by the sum of the mobility of the bulk liquid in the capillary or microchannel, which can be zero or non-zero, and the electrophoretic mobility of the species, determined by the charge on the molecule and the frictional resistance the molecule encounters during migration. For molecules of regular geometry, the frictional resistance is often directly proportional to the size of the molecule, and hence it is common in the art for the statement to be made that molecules are separated by their charge and size. Examples of gels used for electrophoresis include starch, acrylamide, polyethylene oxides, agarose, or combinations thereof. In one embodiment, polyacrylamide gels are used. A gel can be modified by its cross-linking, addition of detergents, or denaturants, immobilization of enzymes or antibodies (affinity electrophoresis) or substrates (zymography) and incorporation of a pH gradient. Examples of capillaries used for electrophoresis include capillaries that interface with an electrospray.

Capillary electrophoresis (CE) is preferred for separating complex hydrophilic molecules and highly charged solutes. Advantages of CE include its use of small sample volumes (sizes ranging from 0.1 to 10 μl), fast separation, reproducibility, ease of automation, high resolution, and the ability to be coupled to a variety of detection methods, including mass spectrometry. CE technology, in general, relates to separation techniques that use narrow bore capillaries, commonly made of fused silica, to separate a complex array of large and small molecules. High voltages are used to separate molecules based on differences in charge, size and/or hydrophobicity. CE technology can also be implemented on microfluidic chips. Depending on the types of capillary and buffers used, CE can be further segmented into separation techniques such as capillary zone electrophoresis (CZE), capillary isoelectric focusing (CIEF), capillary isotachophoresis (cITP) and capillary electrochromatography (CEC). A preferred embodiment to couple CE techniques to electrospray ionization involves the use of volatile solutions, for example, aqueous mixtures containing a volatile acid and/or base and an organic such as an alcohol or acetonitrile.

Capillary isotachophoresis (cITP) is a technique in which the analytes move through the capillary at a constant speed but are nevertheless separated by their respective mobilities. This type of separation is accomplished in a heterogeneous buffer system where the buffers are different upstream and downstream of the sample zone. For a separation of positively-charged analytes, the buffer cation of the first buffer has a mobility and conductivity greater than that of the analytes, and the buffer cation of the second buffer has a mobility and conductivity less than that of the analytes. The voltage gradient per unit length of capillary depends on the conductivity, and therefore the voltage gradient is heterogeneous along the length of the capillary; higher in regions of low conductivity and lower in regions of high conductivity. At steady state, the analytes are focused in zones according to their mobility: if an analyte diffuses into a neighboring zone, it encounters a different field and will either speed up or slow down to rejoin its original zone. An advantage of cITP is that it can be used to concentrate a relatively wide zone of low concentration into a narrow zone of high concentration, thereby improving the limit of detection. Through the appropriate choice of buffers and injected zones, a hybrid separation technique often referred to as transient isotachophoresis-zone electrophoresis (tITP/ZE) can be performed. In tITP/ZE the conditions for isotachophoresis are present only transiently, after which the conditions are set up for zone electrophoresis. In this way, dilute samples can be concentrated and then separated into individual peaks.

Capillary zone electrophoresis (CZE), also known as free-solution CE (FSCE), is one of the simplest forms of CE. The separation mechanism of CZE is based on differences in the electrophoretic mobility of the species, determined by the charge on the molecule, and the frictional resistance the molecule encounters during migration which is often directly proportional to the size of the molecule. The separation typically relies on the charge state of the proteins, which is determined by the pH of the buffer solution.

Capillary isoelectric focusing (CIEF) allows weakly-ionizable amphoteric molecules, such as polypeptides, to be separated by electrophoresis in a pH gradient. A solute migrates to the point in the pH gradient where its net charge is zero. The pH of the solution at the point of zero net charge equals the isoelectric point (pI) of the solute. Because the solute is net neutral at the isoelectric point, its electrophoretic migration is no longer affected by the electric field, and the sample focuses into a tight zone. In CIEF, after all the solutes have focused at their pI's, the bulk solution is often moved past the detector by pressure or chemical means.

CEC is a hybrid technique between traditional liquid chromatography (HPLC) and CE. In essence, CE capillaries are packed with beads (as in traditional HPLC) or a monolith, and a voltage is applied across the packed capillary which generates an electro-osmotic flow (EOF). The EOF transports solutes along the capillary towards a detector. Both chromatographic and electrophoretic separation occurs during their transportation towards the detector. It is therefore possible to obtain unique separation selectivities using CEC compared to both HPLC and CE. The beneficial flow profile of EOF reduces flow related band broadening and separation efficiencies of several hundred thousand plates per meter are often obtained in CEC. CEC also makes it is possible to use small-diameter packings and achieve very high efficiencies.

Chromatography is another type of method for separating a subset of polypeptides, proteins, or other analytes. Chromatography can be based on the differential adsorption and elution of certain analytes or partitioning of analytes between mobile and stationary phases. Liquid chromatography (LC), for example, involves the use of fluid carrier over a non-mobile phase. Conventional analytical LC columns have an inner diameter of roughly 4.6 mm and a flow rate of roughly 1 ml/min. Micro-LC typically has an inner diameter of roughly 1.0 mm and a flow rate of roughly 40 μl/min. Capillary LC generally utilizes a capillary with an inner diameter of roughly 300 μm and a flow rate of approximately 5 μl/min. Nano-LC is available with an inner diameter of 50 μm-1 mm and flow rates of 200 nl/min. Nano-LC can vary in length (e.g., 5, 15, or 25 cm) and have typical packing of C18, 5 μm particle size. In a preferred embodiment, nano-LC is used. Nano-LC provides increased sensitivity due to lower dilution of chromatographic sample. The sensitivity improvement of nano-LC as compared to analytical HPLC is approximately 3700 fold.

In preferred embodiments, the samples are separated using capillary electrophoresis separation, more preferably CEC, or more preferably CZE. This will separate the molecules based on their electrophoretic mobility at a given pH and size (or hydrophobicity in the case of CEC).

In other preferred embodiments, the steps of sample preparation and separation are combined using microfluidics technology. A microfluidic device is a device that can transport fluids containing various reagents such as analytes and elutions between different locations using microchannel structures. Microfluidic devices provide advantageous miniaturization, automation and integration of a large number of different types of analytical operations. For example, continuous flow microfluidic devices have been developed that perform serial assays on extremely large numbers of different chemical compounds.

In a preferred embodiment, microfluidic devices are composed of plastic and formed by means of etching, machining, cutting, molding, casting or embossing. The microfluidics devices may alternatively be made from glass, silicon, or any other material by means of etching, machining, or cutting. The microfluidic devices may be either single use for a single sample; multi-use for a single sample at a time with serial loading; single use with parallel multiple sample processing; multi-use with parallel multiple sample processing; or a combination. Furthermore, more than one microfluidics device may be integrated into the system and can interface with a single detection device.

Once prepared and separated, the analytes are automatically delivered to a detection device, which detects the proteins or other analytes in a sample. In a preferred embodiment, proteins in elutions or solutions are delivered to a detection device by electrospray ionization (ESI). ESI operates by infusing a liquid containing the sample of interest through a channel or needle, which is kept at a potential of typically 1-6 kV, more typically of 1.5-4 kV. The voltage on the needle causes the spray to be charged as it is nebulized. The resultant charged vapor droplets disintegrate and evaporate in a region maintained between atmospheric pressure and a vacuum of several torr, until the solvent is essentially completely stripped off, leaving a charged ion. Alternatively, ions are formed by coulombic ejection from the surface of the droplet, in a process called ion evaporation. In either case, ions are then detected by a detection device such as a mass spectrometer. In a more preferred embodiment, nanospray ionization (NSI) is used. Nanospray ionization is a miniaturized version of ESI and provides low detection limits using extremely small volumes of sample fluid.

In preferred embodiments, separated proteins are directed down a channel that leads to an electrospray ionization emitter, which is built into a microfluidic device (an integrated ESI microfluidic device). Preferably, such an integrated ESI microfluidic device provides the detection device with samples at flow rates and complexity levels that are optimal for detection. Such flow rates are, preferably, approximately 50-approximately 200 μL/min. Furthermore, a microfluidic device is preferably aligned with a detection device for optimal sample capture. See co-pending U.S. application Ser. No. 10/681,742, filed on Jun. 12, 2003. For example, using dynamic feedback circuitry, a microfluidic device may allow for control positioning of an electrospray voltage and for the entire spray to be captured by the detection device orifice. The microfluidic device can be sold separately or in combination with other reagents, software tools and/or devices.

Calibrants can also be sprayed into detection device. Calibrants can be used to set instrument parameters and for signal processing purposes. Calibrants can be utilized before or in parallel with assessment of real sample. Calibrants can interface with a detection device using the same or a separate interface as the samples. In a preferred embodiment, calibrants are sprayed into a detection device using a second interface (e.g., second spray tip) or a second channel on the microfluidic device.

In one embodiment of the invention, the biological sample is not prepared and/or separated on a protein affinity chip.

Identification of Biological Patterns

Detection devices can comprise of any device that is able to detect proteins or other analytes presence and/or level, including for example, NMR, 2-D PAGE technology, Western blot technology, immuno-analysis technology, chromatography, or electrophoresis coupled to spectrophotometric detection either directly or after reaction of eluted products with a detection chemistry, and mass spectrometry. In some preferred embodiments, the methods herein rely on a mass spectrometer to detect marker patterns present in a given sample. There are various forms of mass spectrometers that may be utilized.

In certain embodiments, the methods utilize an ESI-MS detection device. An ESI-MS combines the ESI system with mass spectrometry. Furthermore, an ESI-MS preferably utilizes a time-of-flight (TOF) mass spectrometry system. In TOF-MS, ions are generated by whatever ionization method is being employed, such as ESI, and a voltage potential is applied. The potential extracts the ions from their source and accelerates them towards a detector. By measuring the time it takes the ions to travel a fixed distance, the mass to charge ratio of the ions can be calculated. TOF-MS can be set up to have an orthogonal-acceleration (OA). OA-TOF-MS are advantageous and preferred over conventional on-axis TOF because they have better spectral resolution and duty cycle. OA-TOF-MS also has the ability to obtain spectra, e.g., spectra of proteins and/or protein fragments, at a relatively high speed. In addition to the MS systems disclosed above, other forms of ESI-MS include quadrupole mass spectrometry, ion trap mass spectrometry, Fourier transform ion cyclotron resonance (FTICR-MS), and hybrid combinations of these mass analyzers.

Quadrupole mass spectrometry consists of four parallel metal rods arranged in four quadrants (one rod in each quadrant). Two opposite rods have a positive applied potential and the other two rods have a negative potential. The applied voltages affect the trajectory of the ions traveling down the flight path. Only ions of a certain mass-to-charge ratio pass through the quadrupole filter and all other ions are thrown out of their original path. A mass spectrum is obtained by monitoring the ions passing through the quadrupole filter as the voltages on the rods are varied.

Ion trap mass spectrometry uses three electrodes to trap ions in a small volume. The mass analyzer consists of a ring electrode separating two hemispherical electrodes. A mass spectrum is obtained by changing the electrode voltages to eject the ions from the trap. The advantages of the ion-trap mass spectrometer include compact size, and the ability to trap and accumulate ions to increase the signal-to-noise ratio of a measurement.

FTICR mass spectrometry is a mass spectrometric technique that is based upon an ion's motion in a magnetic field. Once an ion is formed, it eventually finds itself in the cell of the instrument, which is situated in a homogenous region of a large magnet. The ions are constrained in the XY plane by the magnetic field and undergo a circular orbit. The mass of the ion can be determined based on the cyclotron frequency of the ion in the cell.

In a preferred embodiment, the methods herein employ a TOF mass spectrometer, or more preferably, an ESI-TOF-MS, or more preferably an ESI-OA-TOF-MS or more preferably a mass spectrometer having a dual ion funnel to support dynamic switching between multiple quadrupoles in series, the second of which can be used to dynamically filter ions by mass in real time.

The detection device preferably interfaces with a separation/preparation device or microfluidic device, which allows for quick assaying of many of the proteins in a sample, or more preferably, most or all of the proteins in a sample. Preferably, a mass spectrometer is utilized that will accept a continuous sample stream for analysis and provide high sensitivity throughout the detection process (e.g., an ESI-MS). The separation/preparation device can also minimize ion suppression and therefore allow the detection of more proteins.

The detection system utilized preferably allows for the capture and measurement of most or all of the proteins that are introduced into the detection device. It is preferable that one can observe proteins with high information-content that are only present at low concentrations. By contrast, it is preferable to remove those polypeptide or components in advance that are, for example, common to all cells, especially those in high abundance or common in serum.

Analysis of Biological Patterns

The output from a detection device can then be processed, stored, and further analyzed or assayed, e.g., using a bioinformatics system. A bioinformatics system can include one or more of the following: a computer; a plurality of computers connected to a network; a signal processing tool(s); and a pattern recognition tool(s). These tools can be present within the detection device or can be connected to the detection device or can be stand-alone tools into which a user inputs the information obtained from a detection device.

Signal processing utilizes mathematical foundations to align, scale, remove noise from, and reduce the dimensionality of the data. Signal processing may involve any of the following procedures, including alignment, scaling, noise removal, and dimensionality reduction. Dynamic programming or regression methods can be used to align a separation axis with a standard separation profile. Intensities may be normalized, and/or scaled, to allow appropriate comparisons. The data sets can then be transformed using wavelets and/or other mathematical techniques that may be specifically designed for separation and mass spectrometer data to remove noise and leave informative signals. In a preferred embodiment, signal processing filters out noise, leaving informative signals, and reduces spectrum dimensionality.

In some embodiments, signal processing may also involve the calibration of a mass-axis using linear correction determined by the calibrants. Calibration can take place prior to any sample detection; after sample detection; or in recurring intervals, for example.

Following signal processing, pattern recognition tools can be utilized to identify a pattern of subtle differences between phenotypic states. In some preferred embodiments, the pattern is used to make a decision regarding the prostate cancer state of a patient. “Prostate cancer state” is used herein to refer to the status of prostate cancer in the patient being studied. This state can include the absence or the presence of prostate cancer. Also, the various states include different forms of prostate cancer. Also, the prostate cancer state of a patient can be modified based on various treatment regimes being used on the patient. A pattern is obtained by training a pattern recognition algorithm on a sample of the data. The features that comprise the pattern discriminate the subtle differences between phenotypic states. In some embodiments, the data is sampled many times to obtain statistics on the patterns. These statistics and patterns are used to identify markers that constitute the biological pattern. In other embodiments, a metric is calculated, describing the discriminatory power of each point in the data, to identify markers that constitute the biological pattern.

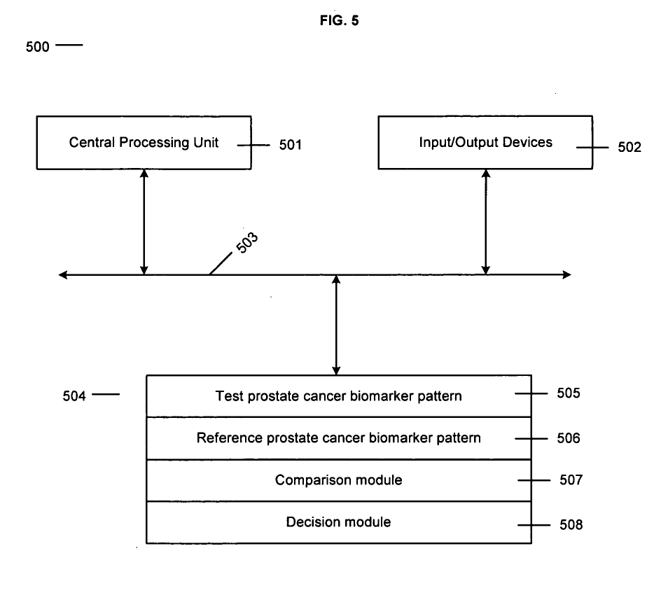

In some embodiments, the methods of the present invention are performed using a computer as depicted in FIG. 5. FIG. 5 illustrates a computer for implementing selected operations associated with the methods of the present invention. The computer 500 includes a central processing unit 501 connected to a set of input/output devices 502 via a system bus 503. The input/output devices 502 may include a keyboard, mouse, scanner, data port, video monitor, liquid crystal display, printer, and the like. A memory 504 in the form of primary and/or secondary memory is also connected to the system bus 503. These components of FIG. 5 characterize a standard computer. This standard computer is programmed in accordance with the invention. In particular, the computer 500 can be programmed to perform various operations of the methods of the present invention, for example, the processing operations of FIGS. 3 and 4.

In some embodiments, the memory 504 of the computer 500 stores test 505 and reference 506 biomarker patterns. The memory 504 also stores a comparison module 507. The comparison module 507 includes a set of executable instructions that operate in connection with the central processing unit 501 to compare the various biomarker patterns. In other words, the comparison module 507 can perform the operation associated with step 304 of FIG. 3 or step 403 of FIG. 4. The executable code of the comparison module 507 may utilize any number of numerical techniques to perform the comparisons.

The memory 504 also stores a decision module 508. The decision module 508 includes a set of executable instructions to process data created by the comparison module 507. The executable code of the decision module 508 may be incorporated into the executable code of the comparison module 507, but these modules are shown as being separate for the purpose of illustration. In preferred embodiments, the decision module 508 includes executable instructions to provide a decision regarding the prostate cancer state of a patient. Preferably, the decision module 508 performs operations associated with step 305 of FIG. 3 or step 404 of FIG. 4.

Patterns of Cancer Markers

In the present invention, patterns of biological markers, specifically prostate cancer markers, are analyzed. Also, novel prostate cancer marker patterns that have been identified are described herein.

In some embodiments, prostate cancer markers are identified in a biological sample from an animal subject and these markers are used to make a decision regarding the prostate cancer state of the subject. Typically, the animal subject is a human patient. Preferably, the markers used in the analysis are characterized by one or more mass spectral signals. Typically, the mass spectral signals are mass spectrum peaks obtained using a mass spectrometry system and are characterized by m/z values, molecular weights, and/or charge states, and/or migration times.

In preferred embodiments, the prostate cancer markers used are characterized by the mass spectral data provided in the following tables. Preferred groups of prostate cancer markers are provided in Table 1. One or more, preferably two or more of the markers of Table 1 are utilized. The markers utilized are those that produce the approximate m/z values in Table 1, assuming the experimental conditions disclosed in the Examples section are utilized, but these makers may be identified according to any other suitable methods.

TABLE 1

| | Separation | | | Separation | | | Separation | |
|---|---|---|---|---|---|---|---|---|
| | time | Levels in | | time | Levels in | | time | Levels in |
| m/z | (seconds) | Cancer | m/z | (seconds) | Cancer | m/z | (seconds) | Cancer |
| 257.1 | 294 | down | 1017.3 | 408 | down | 1023.2 | 384 | down |
| 427.2 | 306 | down | 786.8 | 420 | down | 1034.6 | 582 | down |
| 411.2 | 288 | down | 957.7 | 462 | down | 616.3 | 456 | down |
| 297.1 | 294 | down | 619.2 | 390 | down | 1218.9 | 432 | down |
| 383.1 | 294 | down | 659.1 | 438 | down | 1001.2 | 420 | down |
| 298.1 | 288 | down | 1014.4 | 366 | down | 905.1 | 834 | down |
| 313.1 | 282 | down | 889.3 | 510 | down | 754.8 | 570 | down |
| 425.2 | 294 | down | 673.0 | 498 | down | 1060.3 | 384 | down |
| 258.1 | 288 | down | 960.6 | 348 | down | 591.3 | 600 | up |
| 325.1 | 294 | down | 912.2 | 468 | down | 719.5 | 504 | down |
| 656.9 | 600 | up | 653.1 | 456 | up | 744.6 | 396 | down |
| 269.1 | 282 | down | 1007.9 | 354 | down | 792.4 | 750 | down |
| 702.3 | 456 | up | 1006.5 | 378 | down | 670.9 | 354 | down |
| 255.1 | 282 | down | 1030.4 | 414 | down | 887.9 | 396 | down |
| 698.1 | 408 | down | 950.2 | 462 | down | 629.9 | 516 | down |
| 283.1 | 276 | down | 1061.9 | 426 | down | 753.0 | 408 | down |
| 705.9 | 372 | down | 886.1 | 378 | down | 615.3 | 474 | down |
| 698.4 | 420 | down | 704.4 | 552 | down | 596.9 | 432 | down |
| 1014.0 | 378 | down | 746.0 | 504 | down | 657.7 | 420 | down |
| 706.3 | 372 | down | 691.5 | 390 | down | 745.7 | 402 | down |
| 399.2 | 306 | down | 814.8 | 840 | down | 758.0 | 390 | down |
| 841.5 | 402 | down | 827.3 | 576 | down | 708.1 | 510 | down |
| 842.6 | 456 | up | 301.1 | 282 | down | 1028.0 | 456 | down |
| 747.1 | 390 | down | 1118.8 | 426 | down | 707.3 | 366 | down |
| 811.3 | 432 | up | 855.7 | 390 | down | 751.3 | 504 | down |
| 385.2 | 294 | down | 429.2 | 288 | down | 716.1 | 408 | down |
| 787.4 | 420 | down | 981.8 | 474 | down | 674.7 | 468 | down |
| 677.5 | 630 | up | 835.1 | 366 | down | 658.0 | 432 | down |
| 827.2 | 414 | down | 1046.3 | 408 | down | 634.0 | 426 | down |
| 674.9 | 498 | down | 926.8 | 396 | down | 634.3 | 432 | down |
| 299.1 | 282 | down | 902.3 | 420 | down | 741.0 | 834 | down |
| 529.2 | 360 | down | 1005.8 | 360 | down | 637.5 | 600 | up |
| 921.8 | 408 | down | 758.9 | 492 | down | 896.6 | 600 | down |
| 1011.4 | 390 | down | 864.0 | 420 | down | 759.8 | 504 | down |
| 1085.8 | 402 | down | 361.1 | 282 | up | 894.4 | 402 | down |
| 698.8 | 318 | down | 768.8 | 498 | down | 630.0 | 438 | down |
| 295.1 | 282 | down | 898.8 | 804 | down | 1001.0 | 414 | down |
| 706.6 | 360 | down | 748.0 | 396 | down | 1051.2 | 480 | down |
| 888.4 | 396 | down | 597.1 | 504 | down | 806.7 | 576 | down |
| 706.9 | 360 | down | 1001.9 | 420 | down | 1250.9 | 396 | down |
| 275.1 | 282 | down | 715.2 | 408 | down | 835.6 | 402 | down |
| 928.3 | 402 | down | 752.6 | 402 | down | 745.9 | 420 | down |
| 1017.0 | 390 | down | 828.8 | 384 | down | 679.0 | 498 | down |
| 698.3 | 318 | down | 692.9 | 420 | up | 816.3 | 834 | down |
| 844.7 | 396 | down | 636.4 | 498 | down | 1251.3 | 378 | down |
| 903.0 | 828 | down | 241.1 | 282 | down | 1000.6 | 396 | down |
| 1128.1 | 414 | down | 884.9 | 528 | down | 814.7 | 462 | down |
| 281.1 | 282 | down | 1027.5 | 450 | down | 671.2 | 354 | down |
| 882.8 | 396 | down | 1057.4 | 360 | down | 912.0 | 498 | down |
| 1055.3 | 402 | down | 867.1 | 504 | down | 695.3 | 390 | down |
| 752.3 | 396 | down | 717.3 | 522 | down | 745.9 | 402 | down |
| 886.8 | 432 | down | 822.3 | 384 | down | 658.0 | 414 | down |
| | | | | | | 1007.4 | 360 | down |

An even more preferred set of prostate cancer markers are presented in Table 2.

TABLE 2

| | | Separation | | | |
|---|---|---|---|---|---|
| | | Time | | Charge | Levels in |
| Biomarker | M/Z | (seconds) | MW | State | Cancer |
| 1 | 255.1 | | | | up |
| 2 | 257.1 | 366.00 | 256 | 1 | up |
| 3 | 269.1 | 300.00 | 268 | 1 | up |
| 4 | 295.0 | 300.00 | 294 | 1 | up |
| 5 | 297.0 | 300.00 | 295 | 1 | up |
| 6 | 298.1 | | | | up |
| 7 | 347.1 | | | | up |
| 8 | 361.1 | | | | down |
| 9 | 395.3 | | | | up |
| 10 | 396.2 | | | | up |
| 11 | 405.1 | 300.00 | | | down |
| 12 | 411.2 | | | | up |
| 13 | 419.2 | | | | down |
| 14 | 425.2 | 300.00 | 424.17 | 1 | up |
| 15 | 427.2 | | | | up |
| 16 | 591.2 | 570.00 | 5901.00 | 10 | down |
| 17 | 602.1 | 477.00 | 4209 | 7 | down |
| | 702.3 | 477.00 | 4209 | 6 | down |
| | 842.8 | 477.00 | 4209 | 5 | down |
| 18 | 929.6 | 666.00 | 9287 | 10 | down |
| | 1032.7 | 666.00 | 9287 | 9 | down |
| 19 | 813.4 | 837.00 | 8123 | 10 | up |
| | 903.3 | 837.00 | 8123 | 9 | up |
| | 1016.2 | 837.00 | 8123 | 8 | up |
| | 1161.8 | 837.00 | 8123 | 7 | up |
| 20 | 614.9 | 474.00 | | | up |
| 21 | 810.3 | 513.00 | 13763 | 17 | down |
| | 918.3 | 513.00 | 13763 | 15 | down |
| 22 | 887.9 | 483.00 | 10645 | 12 | up |
| | 968.5 | 483.00 | 10645 | 11 | up |
| | 1065.3 | 483.00 | 10645 | 10 | up |
| 23 | 665.5 | 513.00 | 4655 | 7 | up |
| 24 | 698.1 | 432.00 | 4818 | 7 | up |
| | 813.4 | 432.00 | 4818 | 6 | up |
| 25 | 1143.9 | 618.00 | | 13 | up |

The m/z values provided in the above Tables 1 and 2 are peaks that are obtained for the markers using mass spectrometry system under the conditions disclosed in the Examples section. The markers can have the m/z values in Tables 1 and 2 or a reading +/−0.1 around the m/z location. In another embodiment the peak for a marker in Tables 1 and 2 can be integrated, combining values at several m/z locations that compose the peak. For example, for biomarker 16 in Table 2 at m/z=591.2, all values in the range 591.0 to 591.7 have been integrated. In one embodiment, an user can integrate starting from the closest m/z value to 591.0+/−0.1 and ending at the closest m/z value to 591.7+/−0.1. In yet another embodiment, an algorithm can be used to determine where the peak begins and ends and automatically estimate its integrated area and center location.

A marker may be represented at multiple m/z points in a spectrum. This can be due to the fact that multiple isotopes of the marker are observed and/or that multiple charge states of the marker are observed, or that multiple isoforms of the marker are observed. An example of different isoforms of the same marker is a protein that exists with and without a post-translational modification such as glycoslyation. These multiple representation of a marker can be analyzed individually or grouped together. An example of how multiple representations of a marker may be grouped is that the intensities for the multiple peaks can be summed.

The markers that are characterized by the mass spectral data provided in Tables 1 and 2 above can be identified using different techniques that are known in the art. These techniques are not limited to mass spectrometry systems and include immunoassays, protein chips, multiplexed immunoassays, and complex detection with aptamers and chromatography utilizing spectrophotometric detection.

The markers of Tables 1 and 2 can be further characterized using techniques known in the art. For example, polypeptide markers can be further characterized by sequencing them using enzymes or mass spectrometry techniques. For example, see, Stark, in: Methods in Enzymology, 25:103-120 (1972); Niall, in: Methods in Enzymology, 27:942-1011 (1973); Gray, in: Methods in Enzymology, 25:121-137 (1972); Schroeder, in: Methods in Enzymology, 25:138-143 (1972); Creighton, Proteins: Structures and Molecular Principles (W. H. Freeman, NY, 1984); Niederwieser, in: Methods in Enzymology, 25:60-99 (1972); and Thiede, et al. FEBS Lett., 357:65-69 (1995), Shevchenko, A., et al., Proc. Natl. Acad. Sci. (USA), 93:14440-14445 (1996); Wilm, et al., Nature, 379:466-469 (1996); Mark, J., “Protein structure and identification with MS/MS,” paper presented at the PE/Sciex Seminar Series, Protein Characterization and Proteomics: Automated high throughput technologies for drug discovery, Foster City, Calif. (March, 1998); and Bieman, Methods in Enzymology, 193:455-479 (1990).

In some embodiments, the prostate cancer markers used to make a decision regarding the prostate cancer state of a patient involves the identification of a set of markers. The set can include one or more markers.

Typically, when patterns of prostate cancer markers are used to determine the prostate cancer state, the pattern from a patient, also referred to as test pattern, is compared mathematically to a set of reference patterns. The reference patterns can be derived from the same patient, different patient, or group of patients. In some embodiments, the reference patterns are obtained from normal subjects, i.e. subjects who do not have prostate cancer, as well as from subjects having prostate cancer.

A decision regarding the prostate cancer state of a patient can be made by analyzing a biological sample from a patient for patterns of prostate cancer markers using a mass spectrometry system. In one embodiment, the analysis of the samples does not involve separation on a protein affinity chip and preferably the markers are proteins, protein fragments, peptides, or small molecules. In some preferred embodiments, the samples are prepared and/or separated on a micro-fluidic device and/or delivered to the mass spectrometer by electrospray ionization.

The patterns from a subject suspected of having prostate cancer, in some embodiments, can be compared to reference patterns, which are typically obtained from one or more normal subjects. Also, patterns from the same patient can be compared to each other. Typically, these patterns are obtained at different time points and are used to evaluate the status of prostate cancer in the patient.

In some embodiments, subsets of prostate cancer markers identified herein are used in the classification of prostate cancer states. These subsets can comprise one or more markers described herein. Preferably the subset comprises one marker, preferably about 2 to about 10 markers, more preferable about 10 to about 50 markers, and even more preferably about 50 to about 150 markers.

In other embodiments, the markers described herein are used in combination with known prostate cancer markers. Several prostate cancer markers are known in the art. For example, see Tumor Markers, Physiology, Pathobiology, Technology and Clinical Applications, Editors E. P. Diamandis et al., AACC Press, vol. 36(4), 2003. Examples of known prostate cancer markers that can be used in combination with the markers described herein include, but are not limited to, prostate specific antigen (PSA), human glandular kallikrein 2, acid phosphatase (PAP, ACPP, ACP3), prostate-specific membrane antigen, androgen receptor, and insulin-like growth factors and binding proteins.

In yet other embodiments, the methods described herein are used in combination with known diagnostic techniques for prostate cancer. Examples of other diagnostic techniques include, but are not limited to, digital rectal exam (DRE), prostate biopsy, transrectal ultrasound (TRUS), computed tomography (CT) scan, and magnetic resonance imaging (MRI) scan.

Uses of Markers

In addition to being used for clinical purposes, the markers and patterns of markers have many other applications. The markers identified herein may be entire proteins or fragments of proteins or other analytes. It is intended herein that a particular marker not only encompass the protein fragment, but also the entire parent protein.

The markers and their patterns described herein can be used in the prognosis and treatment of prostate cancer and also in assays to identify and develop novel therapies for prostate cancer. In some embodiments, the biomarkers are used in assays to develop prostate cancer treatments. These treatments include, but are not limited to, antibodies, antisense, and small molecules.

The markers found in the invention can be used to enable or assist in the pharmaceutical drug development process for therapeutic agents for use in prostate cancer. The markers can be used to diagnose disease for patients enrolling in a clinical trail. The markers can indicate the prostate cancer state of patients undergoing treatment in clinical trials, and show changes in the prostate cancer state during the treatment. The markers can demonstrate the efficacy of a treatment, and be used as surrogate endpoints for clinical trial outcome. The markers can be used to stratify patients according to their responses to various therapies.

One embodiment includes antibodies that bind to, and thereby affect the function of, these biomarkers. In other embodiments, cellular expression of the target marker can be modulated, for example, by affecting transcription and/or translation. Suitable agents include anti-sense constructs prepared using antisense technology or gene transcription constructs, such as using RNA interference technology. Also, DNA oligonucleotides can be designed to be complementary to a region of the gene involved in transcription thereby preventing transcription and the production of one or more of the biomarkers. Therapeutic and/or prophylactic polynucleotide molecules can be delivered using gene transfer and gene therapy technologies.

Still other agents include small molecules that bind to or interact with the biomarkers and thereby affect the function thereof, such as an agonist or antagonist, and small molecules that bind to or interact with nucleic acid sequences encoding the biomarkers, and thereby affect the expression of these protein biomarkers. These agents may be administered alone or in combination with other types of treatments known and available to those skilled in the art for treating prostate cancer (e.g., radiation therapy, chemotherapy, hormonal therapy, immunotherapy and anti-tumor agents).

One aspect of the invention is therapeutic agents for use in prostate cancer patients. The therapeutic agents can be used either therapeutically, prophylactically, or both. Preferably, the therapeutic agents have a beneficial effect on the prostate cancer state of a patient. Even more preferably, the markers in Tables 1 and 2 are used as targets for therapeutic agents. For markers that are polypeptides, the therapeutic agents may target the polypeptide or the DNA and/or RNA encoding the polypeptide. The therapeutic agent either directly acts on the markers or modulate other cellular constituents which then have an effect on the markers. In some embodiments, the therapeutic agents either activate or inhibit the activity of the markers. In other embodiments, a marker listed in Tables 1 or 2 is used as the therapeutic or prophylactic agent. In these embodiments, the markers used as the active agent may be be modified to improve certain physical properties in order to improve their therapeutic or prophylactic activities. For example, the marker may be chemically modified to improve bioavailability or to its pharmacokinetic properties.

The prostate cancer therapeutic agents of the present invention can be co-administered with other active pharmaceutical agents that are used for the therapeutic and/or prophylactic treatment of prostate cancer. This co-administration can include simultaneous administration of the two agents in the same dosage form, simultaneous administration in separate dosage forms, and separate administration. For example, the prostate cancer therapeutic agents can be co-administered with chemotherapeutic agents that are used to treat cancer. These two agents can be formulated together in the same dosage form and administered simultaneously. Alternatively, they can be simultaneously administered or separately administered, wherein both the agents are present in separate formulations. In the separate administration protocol, the two agents may be administered a few minutes apart, or a few hours apart, or a few days apart.

The prostate cancer therapeutic agents of the present invention can be used in combination with the other prostate cancer therapies. Examples of prostate cancer therapies include, but are not limited to, surgery, radiation therapy, hormone therapy, and chemotherapy.

The term “treating” as used herein includes having a beneficial effect, i.e., achieving a therapeutic benefit and/or a prophylactic benefit. By therapeutic benefit is meant eradication, amelioration, or prevention of the underlying disorder being treated. For example, in a cancer patient, therapeutic benefit includes eradication or amelioration of the underlying cancer. Also, a therapeutic benefit is achieved with the eradication, amelioration, or prevention of one or more of the physiological symptoms associated with the underlying disorder such that an improvement is observed in the patient, notwithstanding that the patient may still be afflicted with the underlying disorder. For example, administration of prostate cancer therapeutic agents to a patient suffering from prostate cancer provides therapeutic benefit not only when the patient's prostate cancer marker count is decreased, but also when an improvement is observed in the patient with respect to other disorders that accompany prostate cancer like pain and incontinence. For prophylactic benefit, the therapeutic agents may be administered to a patient at risk of developing prostate cancer or to a patient reporting one or more of the physiological symptoms of prostate cancer, even though a diagnosis of prostate cancer may not have been made.

The therapeutic agents of the present invention are administered in an effective amount, i.e., in an amount effective to achieve therapeutic or prophylactic benefit. The actual amount effective for a particular application will depend on the patient (e.g., age, weight, etc.), the condition being treated, and the route of administration. Determination of an effective amount is well within the capabilities of those skilled in the art. The effective amount for use in humans can be determined from animal models. For example, a dose for humans can be formulated to achieve circulating and/or gastrointestinal concentrations that have been found to be effective in animals.

Preferably, the agents used for therapeutic and/or prophylactic benefit can be administered per se or in the form of a pharmaceutical composition. The pharmaceutical compositions comprise the therapeutic agents, one or more pharmaceutically acceptable carriers, diluents or excipients, and optionally additional therapeutic agents. The compositions can be formulated for sustained or delayed release. The compositions can be administered by injection, topically, orally, transdermally, rectally, or via inhalation. Preferably, the therapeutic agent or the pharmaceutical composition comprising the therapeutic agent is administered orally. The oral form in which the therapeutic agent is administered can include powder, tablet, capsule, solution, or emulsion. The effective amount can be administered in a single dose or in a series of doses separated by appropriate time intervals, such as hours.

Pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Suitable techniques for preparing pharmaceutical compositions of the therapeutic agents of the present invention are well known in the art.

Therapeutic and Diagnostic Uses of Patterns of Cancer Markers

The complement of proteins, protein fragments, peptides, or other analytes present at any specific moment in time defines who and what an individual organism is at that moment, as well as the state of health or disease: the biological state. The biological state of a cancer patient reflects not only the presence and nature of the cancer, but the more general state of health and response of the affected individual to the disease.

The methods described herein can be used to identify the state of prostate cancer in a patient, i.e., the prostate cancer state. In one embodiment, the methods are used to detect the earliest stages of disease (e.g. stage I cancer detection). In other embodiments, the methods are used to grade the identified cancer. In one embodiment, the methods are used to diagnose the presence or absence of prostate cancer. The methods can be used to categorize the cancer based on the probability that the cancer will metastasize. Also, these methods can be used to predict the possibility of the cancer going into remission in a particular patient.

In certain embodiments, patients, health care providers, such as doctors and nurses, or health care managers, use the patterns of prostate cancer markers to make a diagnosis, prognosis, and/or select treatment options.

In other embodiments, the methods described herein can be used to predict the likelihood of response for any individual to a particular treatment, select a treatment, or to preempt the possible adverse effects of treatments on a particular individual (e.g. monitoring toxicology due to chemotherapy). Also, the methods can be used to evaluate the efficacy of treatments over time. For example, biological samples can be obtained from a patient over a period of time as the patient is undergoing treatment. The patterns from the different samples can be compared to each other to determine the efficacy of the treatment. Also, the methods described herein can be used to compare the efficacies of different prostate cancer therapies and/or responses to one or more treatments in different populations (e.g., different age groups, ethnicities, family histories, etc.).

In a preferred embodiment, a mass spectrometry system is used to analyze one or more markers of Tables 1 or 2 to evaluate the prostate cancer state of a patient. Intensities for one or more of the markers are obtained from the mass spectrometry system and these intensities are used to make the decision regarding the prostate cancer state. The intensity for a particular maker is normalized and weighted based on the intensity values obtained in samples from previous normal and prostate cancer patients. The normalized and weighted intensities are summed for all the markers being studied and the resulting value is used to make the decision regarding the prostate cancer state. A value greater than zero can indicate, for example, that the patient is healthy and a value less than zero indicates the presence of prostate cancer. In general, the magnitude of the value can be related to the severity grading of the prostate cancer state of the subject.

The following example is intended to illustrate details of the invention, without thereby limiting it in any manner.

### EXAMPLE

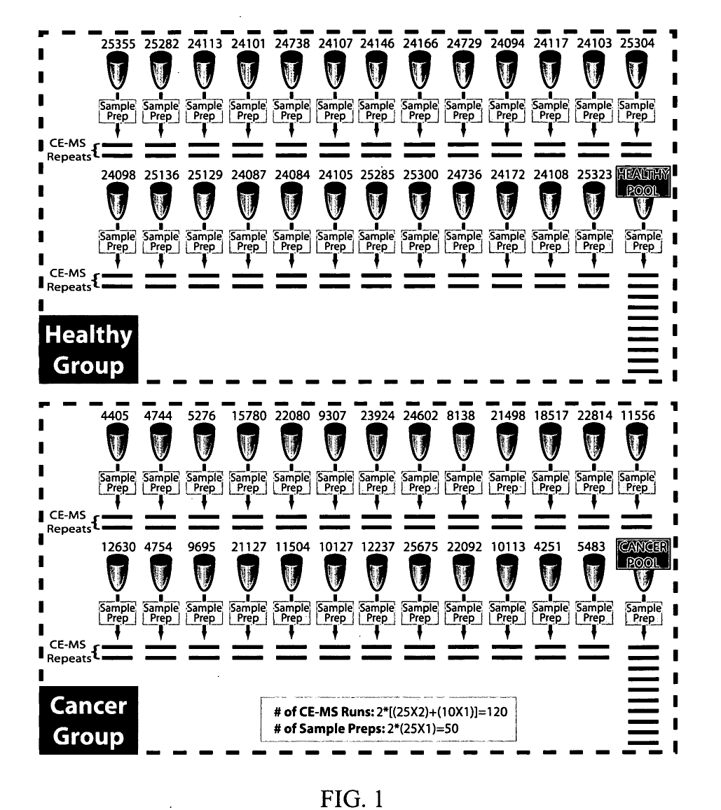

CE-MS was used to identify prostate cancer markers. The experimental design is shown out in FIG. 1. Samples used in this study include 25 serum samples from individuals with prostate cancer and 25 serum samples from individuals without prostate cancer. For each of the 25 prostate cancer and 25 healthy samples, 50 μL was aliquoted and used individually. Bradykinin and ubiquitin were spiked in as pre-processing calibrants such that their concentrations in the sample are 100 nM and 200 nM, respectively.

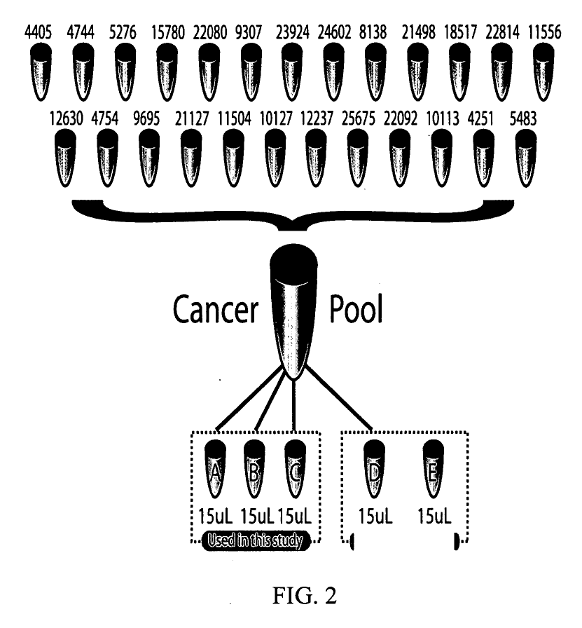

After sample preparation to release carrier protein-bound molecules and to remove high abundance proteins, cancer and healthy pools were created from the sample prepped individual samples and aliquoted as shown in FIG. 2. 3 μL from each prostate cancer sample was pooled with the other prostate cancer samples to form 75 μL of “cancer pool”. 3 aliquots (A, B, C) of 15 μL, each and 2 aliquots (D & E) of 15 μL, each were made of the cancer pool. Aliquots A, B, and C were used in this study.

3 μL from each healthy sample was pooled with the other healthy samples to form 75 μL of “healthy pool”. 3 aliquots (A′, B′, C′) of 15 μL, each and 2 aliquots (D′ & E′) of 15 μL, each were made of the healthy pool. Aliquots A′ B′, and C′ were used in this study.

Aliquoting and adding neurotensin to the pools and individual aliquots were done in one sample “handling”. Neurotensin (the sole post-processing calibrant) was spiked in such that its concentration in the sample is 100 nM. For the pooled samples, neurotensin was added after aliquoting A-E.

After the addition of the post-processing calibrant, each sample was run on the CE-MS multiple times. Individual aliquots were run 2 times. Cancer and healthy pool samples were run 10 times, once each shift. The CE capillary used in the experiments was 50 μm inner diameter x 65 cm long, and coated internally with a positively-charged surface. The run buffer was 20% methanol/60 mM acetic acid, and separation voltage was −30 kV. The sample was stacked in the capillary by transient isotachophoresis, using ammonium ions. The electrospray interface was a sheathless interface, with the electrical contact made at a zero dead volume union, using an electrospray tip distally coated with a conductive surface. Ions were sprayed at the ion source of an orthogonal time-of-flight mass spectrometer that included an ion funnel for high ion transmission. In a direct infusion experiment with a mixture of angiotensin, neurotensin, bradykinin, ubiquitin and lysozyme at 1 nM, for a 3 second acquisition on the mass spectrometer, a signal-to-noise ratio of 10 and a resolution of 4000 for neurotensin in the 3+charge state was observed.

The CE-MS run order was randomized. Before the start of a shift, 3 capillary conditioning runs were performed. Before the 1^{st}, 6^{th}, and after the last run in each shift, peptide standard mixtures were run to track system performance. The “peptide standard mixture” contains the calibrants used in the previous pool/spike serum experiment. Data was then be analyzed as described in the analysis section.

Data Analysis

Two pattern recognition pipelines were used:
a. 1-dimensional: the data at each m/z were integrated across the separation time axis to remove the time dimension, and
b. 2-dimensional: the 2d dataset was considered for pattern recognition.
   For example, in the 1-dimensional analysis, raw data, log-transformed data, raw data normalized by the total ion current, or normalized, transformed data were entered into a feature selection method. The feature selection method iteratively removed features based on the feature weights calculated in a support vector machine or in Fisher discriminant analysis. Once a pre-determined number of features were removed, or once the algorithm converged, the remaining features were used as input into a support vector machine and/or Fisher discriminant analysis. The support vector machine and/or Fisher discriminant analysis methods output a specification of an algorithm by which future data sets can be classified. Generally, this algorithm is a linear or non-linear combination of the feature that were input into the classification algorithm, and generally the evaluation of this combination yields a discriminant score that assigns a class (healthy versus prostate cancer) to a new dataset. This data analysis was performed using 3-fold cross-validation, in which ⅓ of the data was set aside as a test set and the remaining ⅔ of the data was used to generate a classification algorithm. The test set was then used to evaluate the performance of this algorithm. The prostate cancer markers identified using this procedure are listed in Tables 1 and 2.

The intensities obtained from the mass spectrometric analysis are then used to determine the prostate cancer state of the patient. Given intensities of the 25 biomarkers from Table 2 for a given patient sample, two steps are used to calculate the decision function. First, the data is normalized in the following manner:
Normalized biomarker intensity (NBI)=((Biomarker intensity−mean of intensities)/standard deviation of the intensities)

The mean and standard deviations for the 25 biomarkers are provided in Table 3.

TABLE 3

| | | | | | Normalized |
|---|---|---|---|---|---|
| | Intensity | Intensity | Weighted Biomarker | Intensity for | Biomarker Intensity |
| Biomarker | Mean | Standard Deviation | Value | Patient X | for Patient X |
| 1 | 18738.8229067797 | 14623.2352371111 | 0.00287486039568013 | 14723.72 | −0.274570082589529 |
| 2 | 18471.3847711864 | 12668.4350746139 | 0.0152718769589517 | 48474.325 | 2.36832253171791 |
| 3 | 8695.24838998305 | 3927.0012068131 | 0.00209473797462831 | 15227.44 | 1.66340453342276 |
| 4 | 11992.7099237288 | 6345.7415108155 | 0.00078743078942123 | 17371.16 | 0.847568415307228 |
| 5 | 14955.4442372881 | 14060.0906322011 | 0.0207066586062057 | 34681.171 | 1.4029587204463 |
| 6 | 12193.6935084746 | 6350.4687191756 | −0.0219823090617778 | 21756.55 | 1.50585049929462 |
| 7 | 8243.0612372881 | 9690.6255220842 | 0.014879856480702 | 5008.379 | −0.33379498876688 |
| 8 | 5984.7813474576 | 1881.5822857521 | −0.0271510213710407 | 4769.795 | −0.64572586416119 |
| 9 | 10815.2508559322 | 20068.6554836712 | 0.00298210732138475 | 10295.66 | −0.0258906659868153 |
| 10 | 7981.5718728814 | 4096.7590370981 | −0.00604246370468643 | 9582.46 | 0.390769413729697 |
| 11 | 4488.1765593220 | 1274.7329744375 | 0.0336368775785866 | 4197.84 | −0.227762649232606 |
| 12 | 6069.3557033898 | 2001.7760354470 | 0.0237998629569305 | 7420.08 | 0.674762946849112 |
| 13 | 3737.7153050847 | 968.6399624438 | −0.0174155959784781 | 3658.94 | −0.0813256815112163 |
| 14 | 6288.5888559322 | 2074.1374844713 | 0.00312940149063507 | 8610.47 | 1.11944418412538 |
| 15 | 5252.2940593220 | 1673.9265439650 | −0.00453966244716806 | 6454.3 | 0.718075679611864 |
| 16 | 92442.1765084746 | 62259.6895649932 | −0.014386456060012 | 42396.28 | −0.803825024797649 |
| 17 | 503474.4124576270 | 230289.1357501380 | −0.00742832069108851 | 395742.43 | −0.467811831881272 |
| 18 | 371307.3895762710 | 130768.0630903680 | −0.0143882694420193 | 341611.12 | −0.227091148056152 |
| 19 | 691791.9331440670 | 715475.9471252550 | −0.00485154255850317 | 1232029.45 | 0.755074323639501 |
| 20 | 45092.9116694915 | 26668.9120591866 | 0.000702160371199416 | 73987.09 | 1.0834404593027 |
| 21 | 220692.8272881360 | 106758.8838184230 | −0.0177099514748433 | 138803.29 | −0.767051268795718 |
| 22 | 124069.4221440680 | 90114.8949066908 | 0.00317406284571304 | 208228.61 | 0.933909848566925 |
| 23 | 120051.7321186440 | 116826.9117261350 | 0.0119606323436643 | 86102.71 | −0.290592480936474 |
| 24 | 215349.1223728810 | 206287.4667869880 | 0.0071983115720181 | 457247.23 | 1.17262629375784 |
| 25 | 30116.2868050848 | 10673.1143297766 | 0.0293623597760145 | 26958.82 | −0.295833690854026 |

Second this normalized data was used in a decision function to obtain the value L. The normalized intensity for each biomarker is multiplied by the weighted biomarker value for each biomarker and the resulting value for each biomarker is summed. The sum of all the biomarker values are added to a constant to obtain the value L as shown below:
L=(NBI_{—}1*0.00287486039568013+NBI_{—}2*0.0152718769589517+NBI_{—}3* 0.00209473797462831+NBI_{—}4*0.000787430789421225+NBI_{—}5*0.0207066586062057+NBI_{—}6*−0.0219823090617778+NBI_{—}7*0.014879856480702+NBI_{—}8*−0.0271510213710407+NBI_{—}9*0.00298210732138475+NBI_{—}10*−0.00604246370468643+NBI_{—}11*0.0336368775785866+NBI_{—}12*0.0237998629569305+NBI_{—}13*−0.0174155959784781+NBI_{—}14*0.00312940149063507+NBI_{—}15*−0.00453966244716806+NBI_{—}16*−0.014386456060012+NBI_{—}17*−0.00742832069108851+NBI_{—}18*−0.0143882694420193+NBI_{—}19*−0.00485154255850317+NBI_{—}20*0.000702160371199416+NBI_{—}21*−0.0177099514748433+NBI_{—}22*0.00317406284571304+NBI_{—}23* 0.0119606323436643+NBI_{—}24*0.0071983115720181+NBI_{—}25*0.0293623597760145)−0.00127399357851983

The value of L determines the prostate cancer state of a patient. If L>0 then the patient is classified as having prostate cancer, otherwise the patient is classified as healthy.

For example, one of the samples from the 50 samples was used as a test case. Data for this patient including biomarker intensity and normalized data is included in Table 3. The normalized biomarker intensity is used to obtain the L value of 0.0826. As this value is greater than 0.0, this patient was classified as having prostate cancer.

The above examples are in no way intended to limit the scope of the invention. Further, it can be appreciated to one of ordinary skill in the art that many changes and modifications can be made thereto without departing from the spirit or scope of the appended claims, and such changes and modifications are contemplated within the scope of the instant invention.

All publications, patents, and patent applications mentioned in this specification are herein incorporated by reference to the same extent as if each individual publication, patent, or patent application was specifically and individually indicated to be incorporated by reference.

1. (canceled)

2. A method of analyzing prostate cancer states comprising:
identifying a first subset of prostate cancer markers in a biological sample, wherein said markers in said first subset comprise markers from a first set of prostate cancer markers, said markers in said first set being those markers that can provide mass spectral signals selected from following approximate m/z values:

| Biomarker | M/Z |
|---|---|
| 1 | 255.1 |
| 2 | 257.1 |
| 3 | 269.1 |
| 4 | 295.0 |
| 5 | 297.0 |
| 6 | 298.1 |
| 7 | 347.1 |
| 8 | 361.1 |
| 9 | 395.3 |
| 10 | 396.2 |
| 11 | 405.1 |
| 12 | 411.2 |
| 13 | 419.2 |
| 14 | 425.2 |
| 15 | 427.2 |
| 16 | 591.2 |
| 17 | 602.1 |
| | 702.3 |
| | 842.8 |
| 18 | 929.6 |
| | 1032.7 |
| 19 | 813.4 |
| | 903.3 |
| | 1016.2 |
| | 1161.8 |
| 20 | 614.9 |
| 21 | 810.3 |
| | 918.3 |
| 22 | 887.9 |
| | 968.5 |
| | 1065.3 |
| 23 | 665.5 |
| 24 | 698.1 |
| | 813.4 |
| 25 | 1143.9 |

and
making a decision regarding a prostate cancer state.

3. The method of claim 2 wherein said markers in said first subset are further characterized by following approximate molecular weights and charge states:

| | | | Charge |
|---|---|---|---|
| Biomarker | M/Z | MW | State |
| 2 | 257.1 | 256 | 1 |
| 3 | 269.1 | 268 | 1 |
| 4 | 295.0 | 294 | 1 |
| 5 | 297.0 | 295 | 1 |
| 14 | 425.2 | 424.17 | 1 |
| 16 | 591.2 | 5901.00 | 10 |
| 17 | 602.1 | 4209 | 7 |
| | 702.3 | 4209 | 6 |
| | 842.8 | 4209 | 5 |
| 18 | 929.6 | 9287 | 10 |
| | 1032.7 | 9287 | 9 |
| 19 | 813.4 | 8123 | 10 |
| | 903.3 | 8123 | 9 |
| | 1016.2 | 8123 | 8 |
| | 1161.8 | 8123 | 7 |
| 21 | 810.3 | 13763 | 17 |
| | 918.3 | 13763 | 15 |
| 22 | 887.9 | 10645 | 12 |
| | 968.5 | 10645 | 11 |
| | 1065.3 | 10645 | 10 |
| 23 | 665.5 | 4655 | 7 |
| 24 | 698.1 | 4818 | 7 |
| | 813.4 | 4818 | 6 |
| 25 | 1143.9 | | 13 |

4. The method of claim 2 wherein said m/z values of said markers are obtained in an orthogonal time-of-flight mass spectrometer, wherein said mass spectrometer provides a signal-to-noise ratio of 10 and a resolution of 4000 for neurotensin in the 3+charge state in a direct infusion experiment with a mixture of angiotensin, neurotensin, bradykinin, ubiquitin and lysozyme at 1 nM, for a 3 second acquisition.

5. The method of claim 2 wherein said first subset comprises at least one marker from said first set of markers.

6. The method of claim 2 wherein said first subset comprises at least five markers from said first set of markers.

7. The method of claim 2 wherein said first subset comprises about 5 to about 10 markers from said first set of markers.

8. The method of claim 2 wherein said first subset comprises about 15 to about 25 markers from said first set of markers.

9. The method of claim 2 wherein said first subset comprises about 30 to about 50 markers from said first set of markers.

10. The method of claim 2 further comprising:
identifying a second subset of prostate cancer markers in a biological sample from a second set of prostate cancer markers, said second set comprising at least one marker selected from prostate specific antigen, human glandular kallikrein 2, acid phosphatase, prostate-specific membrane antigen, androgen receptor, insulin-like growth factor, and insulin-like growth factor binding protein.

11. The method of claims 2 wherein said markers are proteins, peptides, and/or protein fragments.

12. The method of claims 2 wherein said markers are identified using a mass spectrometry system.

13. The method of claim 12 wherein said mass spectrometry system is a time-of-flight mass spectrometry system.

14. The method of claim 12 wherein said biological sample is separated using capillary electrophoresis, reverse phase liquid chromatography, and microchannel electrophoresis or capillary electrophoresis on a chip format.

15-16. (canceled)

17. The method of claim 12 wherein said biological sample is prepared and/or separated on a microfluidics device.

18. The method of claim 12 wherein said biological sample is delivered to said mass spectrometry system by electrospray ionization.

19. The method of claim 12 wherein said biological sample is delivered to said mass spectrometry system by matrix assisted laser desorption ionization.

20. The method of claim 12 wherein said biological sample is not prepared and/or separated on a protein affinity chip.

21. The method of claim 2 wherein said markers are identified using at least one technique selected from an antibody-based technique, a multiplexed antibody, a protein affinity chip, an aptamer, and a microsequencing technique.

22. The method of claim 2 wherein said markers are identified using chromatography and electrophoresis followed by spectrophotometric detection of eluting analytes with or without the application of a detection chemistry.

23. The method of claim 2 further comprising:
comparing said first subset of prostate cancer markers with a second subset of prostate cancer markers wherein said first subset is obtained from a normal biological sample and said second subset is obtained from a biological sample of a putative prostate cancer patient.

24. The method of claim 2 further comprising:
selecting a treatment or performing a diagnostic assay based on said decision regarding a prostate cancer state.

25. A diagnostic product for prostate cancer comprising a set of components wherein at least one of said components from said set of components is adapted and configured for performing the method as recited in claim 2.

26. A method of analyzing prostate cancer states in an animal subject comprising:
obtaining a biological sample from a first animal subject;
detecting a test pattern of prostate cancer markers in said sample using a detection device, wherein said detection device is a mass spectrometer and said sample is not prepared and/or separated on a protein affinity chip;
comparing said test pattern of prostate cancer markers with a reference pattern, wherein said reference pattern is a pattern of prostate cancer markers from a reference sample, said reference sample being obtained from said first animal subject or from a second animal subject;
making a decision regarding said prostate cancer state in said first animal subject based on differences and/or similarities between said test pattern and said reference pattern, wherein said prostate cancer markers are not glycolipids or oligosaccharides.

27. The method of claim 26 wherein said first animal subject is a putative prostate cancer patient and said second animal subject is a normal subject or a prostate cancer patient.

28. (canceled)

29. The method of claim 26 further comprising:
preparing and/or separating said sample for analysis on a microfluidics device; and
delivering said sample by electrospray ionization to said detection device.

30-33. (canceled)

34. A method of analyzing prostate cancer states comprising:
reviewing results of a comparison of patterns of prostate cancer markers; said comparison being performed between a test pattern and a reference pattern, said test pattern being a pattern of prostate cancer markers from a first biological sample and said reference pattern being a pattern of prostate cancer markers from a second biological sample, said patterns being obtained using a detection device, wherein said detection device is a mass spectrometer and said samples are not prepared and/or separated on a protein affinity chip; and
making a decision regarding a prostate cancer therapy to be used for a patient, said decision being based on said comparison between said test and reference patterns, wherein said prostate cancer markers are not glycolipids or oligosaccharides.

35. The method of claim 26 or 34 wherein said markers of said test and/or reference pattern can provide mass spectral signals selected from following approximate

| Biomarker | M/Z |
|---|---|
| 1 | 255.1 |
| 2 | 257.1 |
| 3 | 269.1 |
| 4 | 295.0 |
| 5 | 297.0 |
| 6 | 298.1 |
| 7 | 347.1 |
| 8 | 361.1 |
| 9 | 395.3 |
| 10 | 396.2 |
| 11 | 405.1 |
| 12 | 411.2 |
| 13 | 419.2 |
| 14 | 425.2 |
| 15 | 427.2 |
| 16 | 591.2 |
| 17 | 602.1 |
| | 702.3 |
| | 842.8 |
| 18 | 929.6 |
| | 1032.7 |
| 19 | 813.4 |
| | 903.3 |
| | 1016.2 |
| | 1161.8 |
| 20 | 614.9 |
| 21 | 810.3 |
| | 918.3 |
| 22 | 887.9 |
| | 968.5 |
| | 1065.3 |
| 23 | 665.5 |
| 24 | 698.1 |
| | 813.4 |
| 25 | 1143.9 |

m/z values:

36. The method of claim 35 wherein said markers are further characterized by following approximate molecular weights and charge states:

| | | | Charge |
|---|---|---|---|
| Biomarker | M/Z | MW | State |
| 2 | 257.1 | 256 | 1 |
| 3 | 269.1 | 268 | 1 |
| 4 | 295.0 | 294 | 1 |
| 5 | 297.0 | 295 | 1 |
| 14 | 425.2 | 424.17 | 1 |
| 16 | 591.2 | 5901.00 | 10 |
| 17 | 602.1 | 4209 | 7 |
| | 702.3 | 4209 | 6 |
| | 842.8 | 4209 | 5 |
| 18 | 929.6 | 9287 | 10 |
| | 1032.7 | 9287 | 9 |
| 19 | 813.4 | 8123 | 10 |
| | 903.3 | 8123 | 9 |
| | 1016.2 | 8123 | 8 |
| | 1161.8 | 8123 | 7 |
| 21 | 810.3 | 13763 | 17 |
| | 918.3 | 13763 | 15 |
| 22 | 887.9 | 10645 | 12 |
| | 968.5 | 10645 | 11 |
| | 1065.3 | 10645 | 10 |
| 23 | 665.5 | 4655 | 7 |
| 24 | 698.1 | 4818 | 7 |
| | 813.4 | 4818 | 6 |
| 25 | 1143.9 | | 13 |

37-44. (canceled)

45. A prostate cancer therapeutic agent wherein said therapeutic agent has a beneficial effect on a prostate cancer state and said therapeutic agent targets at least one prostate cancer marker, wherein said marker is selected from a set of prostate cancer markers that can provide mass spectral signals selected from following approximate m/z values:

| Biomarker | M/Z |
|---|---|
| 1 | 255.1 |
| 2 | 257.1 |
| 3 | 269.1 |
| 4 | 295.0 |
| 5 | 297.0 |
| 6 | 298.1 |
| 7 | 347.1 |
| 8 | 361.1 |
| 9 | 395.3 |
| 10 | 396.2 |
| 11 | 405.1 |
| 12 | 411.2 |
| 13 | 419.2 |
| 14 | 425.2 |
| 15 | 427.2 |
| 16 | 591.2 |
| 17 | 602.1 |
| | 702.3 |
| | 842.8 |
| 18 | 929.6 |
| | 1032.7 |
| 19 | 813.4 |
| | 903.3 |
| | 1016.2 |
| | 1161.8 |
| 20 | 614.9 |
| 21 | 810.3 |
| | 918.3 |
| 22 | 887.9 |
| | 968.5 |
| | 1065.3 |
| 23 | 665.5 |
| 24 | 698.1 |
| | 813.4 |
| 25 | 1143.9 |

46. The prostate cancer therapeutic agent of claim 43 wherein said markers are further characterized by following approximate molecular weights and charge states:

| | | | Charge |
|---|---|---|---|
| Biomarker | M/Z | MW | State |
| 2 | 257.1 | 256 | 1 |
| 3 | 269.1 | 268 | 1 |
| 4 | 295.0 | 294 | 1 |
| 5 | 297.0 | 295 | 1 |
| 14 | 425.2 | 424.17 | 1 |
| 16 | 591.2 | 5901.00 | 10 |
| 17 | 602.1 | 4209 | 7 |
| | 702.3 | 4209 | 6 |
| | 842.8 | 4209 | 5 |
| 18 | 929.6 | 9287 | 10 |
| | 1032.7 | 9287 | 9 |
| 19 | 813.4 | 8123 | 10 |
| | 903.3 | 8123 | 9 |
| | 1016.2 | 8123 | 8 |
| | 1161.8 | 8123 | 7 |
| 21 | 810.3 | 13763 | 17 |
| | 918.3 | 13763 | 15 |
| 22 | 887.9 | 10645 | 12 |
| | 968.5 | 10645 | 11 |
| | 1065.3 | 10645 | 10 |
| 23 | 665.5 | 4655 | 7 |
| 24 | 698.1 | 4818 | 7 |
| | 813.4 | 4818 | 6 |
| 25 | 1143.9 | | 13 |

47. A method of treating prostate cancer comprising administering to a subject in need thereof an effective amount of said therapeutic agent of claim 45.
